(19) United States
(12) Patent Application Publication
(10) Pub. No.: US 2022/0277451 A1
(43) Pub. Date: Sep. 1, 2022

(54) SYSTEMS, METHODS AND APPARATUSES FOR VISUALIZATION OF IMAGING DATA

(71) Applicant: Perimeter Medical Imaging Inc., Toronto (CA)

(72) Inventors: Vladimir PEKAR, Toronto, Ontario (CA); David REMPEL, Toronto, Ontario (CA)

(21) Appl. No.: 17/632,007

(22) PCT Filed: Jul. 31, 2020
(86) PCT No.: PCT/CA2020/051057

§ 371 Date: Feb. 1, 2022

Related U.S. Application Data
(60) Provisional application No. 62/881,579, filed on Aug. 1, 2019.

(51) Int. Cl. G06T 7/00; G06T 5/00; G06T 5/50; G06F 3/04845; G16H 30/40; G16H 50/20

(52) U.S. Cl. CPC G06T 7/0014; G06T 5/005; G06T 5/50; G06F 3/04845; G16H 30/40; G16H 50/20; G06T 2200/24; G06T 2207/30204; G06T 2207/10024; G06T 2207/20081; G06T 2207/30096; G06T 2207/10101; G06T 2207/20084

(57) ABSTRACT

Medical imaging data can include imaging data of tissue portions, such as, for example, tissue samples. A processor is configured to receive imaging data obtained from imaging tissue. The imaging data includes a two-dimensional scan of tissue, such as, for example, a B-scan. The processor is configured to analyze the imaging data, and to identify imaging data associated with suspicious areas. The processor is configured to generate a visual interface that displays the imaging data associated with the suspicious areas in a stitched view. The processor, via the visual interface, enables a user to navigate to locations in the two-dimensional scan that correspond to imaging data associated with suspicious areas that the user determines to be positive for a characteristic (e.g., cancer).

### CROSS-REFERENCE TO RELATED APPLICATIONS

This application claims priority to and the benefit of U.S. Provisional Patent Application No. 62/881,579, filed Aug. 1, 2019, titled “SYSTEMS, METHODS AND APPARATUSES FOR VISUALIZATION OF IMAGING DATA,” and PCT Application WO2021/016721 filed Jul. 31, 2020, titled “SYSTEMS, METHODS AND APPARATUSES FOR VISUALIZATION OF IMAGING DATA,” the disclosures of which are incorporated by reference herein.

### TECHNICAL FIELD

The embodiments described herein relate to methods and apparatus for visualization of imaging data of tissue. More specifically, systems, methods, and apparatuses described herein provide a visual user interface for viewing of medical imaging data.

### BACKGROUND

Some common medical imaging applications involve generation of large amounts of imaging data that is difficult, tedious, and/or time consuming to review or curate manually. Thus, a need exists for improved systems, apparatuses, and methods for efficient review of imaging data.

### SUMMARY

In some embodiments, an apparatus includes a memory and a processor operatively coupled to the memory. The processor can be configured to receive imaging data obtained from an imaging device imaging tissue. In some embodiments, the imaging data can include two-dimensional scans of tissue, such as, for example, optical coherence tomography (OCT) B-scans. The processor can be configured to analyze the imaging data, and to identify imaging data associated with diagnostically relevant areas. The processor can be configured to generate a visual interface that displays the imaging data associated with the diagnostically relevant areas in a two-dimensional stitched view. In some embodiments, the view can be a stitched panoramic view. The processor, via the visual interface, can enable a user to navigate to locations in the original imaging data that correspond to imaging data associated with diagnostically relevant areas that the user determines to be positive for a characteristic (e.g., cancer).

### BRIEF DESCRIPTION OF THE DRAWINGS

FIG. 1 is an example B-scan of a wide-field OCT dataset of a tissue portion related to ductal carcinoma in situ (DCIS).

FIG. 2 is a schematic illustration of a compute device configured to provide visualization of imaging data, according to an embodiment.

FIG. 3A is a flowchart depicting a method of processing and presenting imaging data, according to an embodiment.

FIG. 3B is a flowchart depicting a method of visualizing imaging data, according to an embodiment.

FIG. 4 is an example of a stitched view of imaging data flagged as suspicious for DCIS, according to an embodiment.

FIG. 5 is an example user interface including different areas for displaying a stitched view of diagnostically relevant imaged patches of a tissue portion taken from a volume of imaging data, a view of the volume of imaging data at a set depth, and a perspective view of the tissue portion, according to an embodiment.

FIG. 6 is an example user interface showing a stitched view of diagnostically relevant imaged patches of a tissue portion taken from a plurality of locations in a scanned volume of the tissue portion, according to an embodiment.

FIG. 7 is an example user interface showing a two-dimensional scan of tissue with portions of the scan that correspond to patches of diagnostically relevant imaged areas flagged, according to an embodiment.

### DETAILED DESCRIPTION

Systems, methods, and apparatuses described herein relate to visualization of imaging data of tissue. In embodiments, systems, methods, and apparatuses described herein provide a visual user interface for viewing imaging data of tissue.

Wide-field imaging techniques enable scanning of surgically excised tissue. For example, imaging systems as described in International Patent Application No. PCT/CA2018/050874, published as International Patent Application Publication No. WO 2019/014767 (“the '767 Publication”), filed Jul. 18, 2018, titled “Sample container for stabilizing and aligning excised biological tissue samples for ex vivo analysis,” incorporated herein by reference, enable scanning of large areas of surgically excised tissue at high resolution. Such imaging techniques and systems provide opportunities for intraoperative assessment of surgical margins in a variety of clinical applications, e.g., in breast-conserving surgical treatment for breast cancer in which patients may undergo multiple surgeries due to imprecise intraoperative tumor margin assessment.

Wide-field imaging techniques, however, typically generate large datasets of images, which makes their interactive visual review in a clinical setting challenging and time-consuming Systems, methods, and apparatuses described herein provide a visual tool for facilitating efficient review and analysis of large datasets. Such review and analysis of imaging data can be used in several applications, including, for example, diagnosis, research, technology development, etc.

Biological tissue is frequently imaged in one, two, and/or three dimensions to evaluate properties of the tissue to characterize the tissue. In some instances, the characterization of the tissue is used to diagnose health conditions of the origin of the tissue. OCT, one example imaging technique, is a non-invasive imaging technique that renders cross-sectional views of a three-dimensional tissue portion. Wide-field OCT imaging technique enables efficient scanning of large areas of surgically excised tissue (e.g., breast tissue) with high resolution to make a medical diagnosis, for example, a diagnosis of DCIS.

When using such imaging techniques for diagnostic purposes, e.g., in detecting areas suspicious for DCIS, it can be important to have visualization systems that enable high speed and accurate review of imaging data. Since imaging techniques such as wide-field OCT provide new opportunities for intraoperative assessment of tissue in a variety of clinical applications, it can be important that such assessment is accurate and does not lead to unnecessary treatment. For example, to treat primary breast cancer, a patient may opt for a breast-conserving surgical treatment to remove malignant tissue. However, if imprecisely and/or inaccurately diagnosed at the margin, the patient may be required to undergo repeat surgeries to treat the breast cancer.

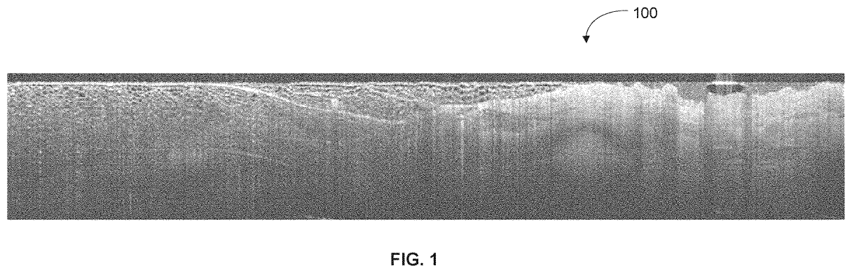

Imaging techniques, such as wide-field OCT, typically generate large datasets. For example, a dataset acquired by OCT of a volume of tissue can include several B-scans or cross-sectional slices of the tissue. In some instances, the number of B-scans can lie in the range of 400-600, which makes their visual review challenging. Existing methods of reviewing such OCT data include visual assessment of each B-scan of an acquired tissue volume with the purpose of detecting certain visual cues that have known associations with malignant tissue via pathological evaluation studies. For example, FIG. 1 depicts a B-scan 100 of a wide-field OCT dataset of a tissue portion (e.g., a core of tissue) acquired for evaluating DCIS. The visible features of the tissue depicted in FIG. 1 are typically linked to DCIS. With the large number of B-scans, however, it can be tedious and time-consuming to review all of the B-scans of the tissue. Applying algorithms of computer-assisted detection (CAD) can potentially reduce the amount of imaging data and therefore make the review process more user-friendly. Even with such systems, the efficient presentation of the results generated by the algorithm to the user remains important.

For example, a CAD algorithm can be programmed to detect suspicious areas in imaging data, e.g., in the B-scans. One method of presenting the results of a CAD algorithm can be to highlight the suspicious areas in individual B-scans of the image data when a user scrolls through the B-scans during his visual review. For instance, the suspicious areas can be marked by color, boxes, arrows, transparent overlays, etc. Such method, while drawing attention to suspicious areas, can still be rather inefficient, especially when a CAD algorithm produces false positive detections.

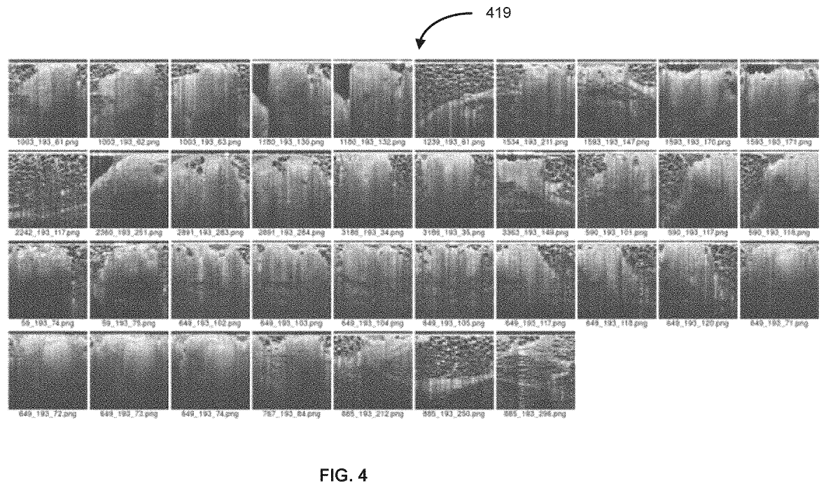

Systems, methods, and apparatuses disclosed herein provide a visual user interface that is configured to reduce the time required for review of imaging data and/or scanning OCT volumes. Instead of highlighting individual areas detected by a CAD algorithm as being suspicious in individual B-scans, such systems, methods, and apparatuses can generate a consolidated or combined view of portions of the image data (e.g., patches of fixed size) that have been identified as suspicious by the CAD algorithm. In some embodiments, this consolidated view can be a stitched view, e.g., a stitched panoramic view. The consolidated view can reduce the amount of image data that needs to be required to a few pages of combined data. Further details of such a view are described below with reference to FIG. 4.

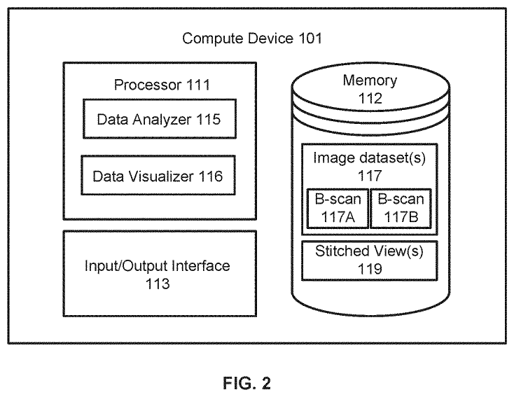

FIG. 2 is a schematic illustration of an example compute device 101 that can be configured to present image data, according to embodiments described herein. While the compute device is depicted as a single device, it can be appreciated that any number of compute devices can collectively operate to perform the functions of the compute device 101. The compute device 101 can be or form part of a system that includes other components for imaging and/or visualizing a tissue sample, e.g., the imaging system described in the '767 Publication, incorporated by reference above.

The compute device 101 can be a hardware-based computing device and/or a multimedia device, such as, for example, a server, a desktop compute device, a smartphone, a tablet, a wearable device, a laptop and/or the like. The compute device 101 includes a processor 111, a memory 112 (e.g., including data storage), and an input/output interface 113.

The processor 111 can be, for example, a hardware-based integrated circuit (IC) or any other suitable processing device configured to run and/or execute a set of instructions or code associated with presenting image data. For example, the processor 111 can be a general purpose processor, a central processing unit (CPU), an accelerated processing unit (APU), an application specific integrated circuit (ASIC), a field programmable gate array (FPGA), a programmable logic array (PLA), a complex programmable logic device (CPLD), a programmable logic controller (PLC) and/or the like. The processor 111 can be operatively coupled to the memory 112 through a system bus (for example, address bus, data bus and/or control bus). As depicted in FIG. 2, the processor 111 can be configured to execute modules, processes, and/or functions illustrated as data analyzer 115 and data visualizer 116, further described below.

The memory 112 of the compute device 101 can be, for example, a random access memory (RAM), a memory buffer, a hard drive, a read-only memory (ROM), an erasable programmable read-only memory (EPROM), and/or the like. The memory 112 can store, for example, one or more software modules and/or code that can include instructions to cause the processor 111 to perform one or more processes, functions, and/or the like (e.g., relating to a CAD algorithm and/or presenting image data). In some embodiments, the memory 112 can include extendable storage units that can be added and used incrementally. In some implementations, the memory 112 can be a portable memory (for example, a flash drive, a portable hard disk, and/or the like) that can be operatively coupled to the processor 111. In other instances, the memory 112 can be remotely situated and coupled to the processor 111 of the compute device 101. For example, a remote database server can serve as the memory 112 and be operatively coupled to the processor 111 of the compute device 101.

In some embodiments, the memory 112 can be configured to store imaging dataset(s) 117 including B-scans 117A-B. In some embodiments, the dataset(s) 117 can represent volumes of biological tissue portions imaged using a wide-field OCT imaging system, e.g., as described in the '767 Publication. In some embodiments, the memory 112 can be configured to store consolidated views or visual representations 119 (e.g., stitched views) of patches of the imaging data associated with anomalous or suspicious areas of tissue.

The input/output interface 113 can be operatively coupled to the processor 111 and memory 112. The input/output interface 113 can include, for example, a network interface card (NIC), a Wi-Fi™ module, a Bluetooth® module and/or any other suitable wired and/or wireless communication device. Furthermore the input/output interface 113 can include a switch, a router, a hub and/or any other network device. The input/output interface 113 can be configured to connect the compute device 101 to a communication network such as, for example, the Internet, an intranet, a local area network (LAN), a wide area network (WAN), a metropolitan area network (MAN), a worldwide interoperability for microwave access network (WiMAX®), an optical fiber (or fiber optic)-based network, a Bluetooth® network, a virtual network, and/or any combination thereof.

In some instances, the input/output interface 113 can facilitate receiving and/or transmitting data (e.g., raw imaging data sets, analyzed imaging data sets, image patches or stitched views of image patches associated with suspicious areas, etc.) through a communication network, e.g., to an external compute device (e.g., a mobile device such as a smart phone, a local computer, and/or a remote server). In some instances, received data can be processed by the processor 111 and/or stored in the memory 112 as described in further detail herein. In some instances, the input/output interface 113 can be configured to send data analyzed by processor 111 to an external compute device such that the external compute device can further provide visualization of the image data and/or analyze such data. In some embodiments, the input/output interface 113 can be configured to periodically connect, e.g., 10 times per day, to an external device to log data stored in the onboard memory. In some embodiments, the input/output interface 113 can be activated on demand by a user to send and/or receive data from an external compute device.

In some embodiments, the input/output interface 113 can include a user interface that can be configured to receive inputs and/or send outputs to a user operating the compute device 111. The user interface can include, for example, a display device (e.g., a display, a touch screen, etc.), an audio device (e.g., a microphone, a speaker), and optionally one or more additional input/output device(s) configured for receiving an input and/or generating an output to a user.

The processor 111, operating as data analyzer 115, can be configured to receive imaging data (e.g., image dataset(s) 117) associated with tissue (e.g., a tissue sample such as, for example, a core of tissue), and process and/or analyze that imaging data to detect anomalous or suspicious areas in the imaged tissue. For example, the data analyzer 115 can be configured to receive raw imaging data and to parse that imaging data into B-scans or patches (e.g., portions of a B-scan). In some embodiments, the data analyzer 115 can be configured to process (e.g., filter, transform, etc.) the imaging data. In some embodiments, the imaging data provided to data analyzer 115 can come pre-processed, such that the data analyzer 115 does not need to further process the imaging data before analysis.

The data analyzer 115 can be configured to analyze portions of the imaging data (e.g., pixels, lines, slices, voxels, etc.) to detect anomalous or suspicious areas in the imaging data. In some implementations, the data analyzer 115 can use CAD algorithms to identify areas (e.g., patches of a fixed size) of scanned image data that capture anomalous or suspicious features. For example, the CAD algorithms can be algorithms that have been trained or calibrated using image datasets of benign and malignant tissue such that the algorithms are capable of identifying suspicious areas in the tissue that includes features similar to previously identified malignant tissue. In some embodiments, the CAD algorithms can be configured to use one or more analytical tools, such as, for example, a convolutional neural network model, a statistical model, machine learning techniques, or any other suitable tools, to perform the detection of suspicious or anomalous areas. For example, in some implementations, a computer algorithm, based on a convolutional neural network, can be trained to differentiate between the malignant and benign patches. The patches that are identified by the algorithm as being suspicious can be passed to the data visualizer 116, e.g., for generating a stitched view as further described below.

In some embodiments, the data analyzer 115 can identify in the imaging data where areas of anomalous or suspicious data has been detected. For example, the data analyzer 115 can use a suitable annotation tool (e.g., colored marking, arrows, outline, etc.) to flag or mark portions of the imaging data detected to include suspicious features. In some embodiments, the data analyzer 115 can generate a dataset including patches of imaged areas having fixed width that include suspicious areas, and pass this dataset onto the data visualizer 116. In some embodiments, the data analyzer 115 can make in each patch where suspicious features were identified (e.g., a portion of a lesion) that led to each patch being identified as suspicious.

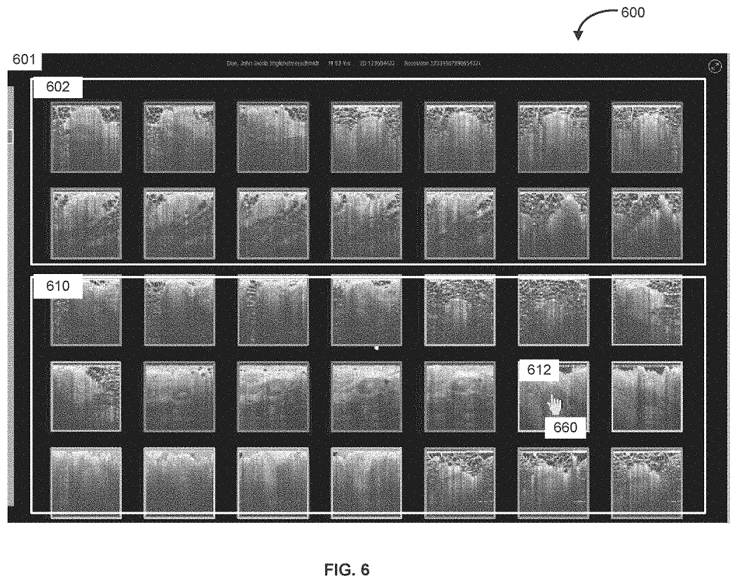

The processor, operating as data visualizer 116, can be configured to receive the analyzed data from the data analyzer 115 and generate one or more consolidated view(s) (e.g., stitched view(s) 119) of those portions of the image data (e.g., patches) that include detected suspicious areas. In some implementations, the data visualizer 116 can generate a stitched view (e.g., a stitched panoramic view) of the patches including the suspicious areas, as further described with reference to FIG. 4. The consolidated views or visual representations can include the patches with the suspicious areas arranged according to a predefined layout (e.g., in a grid pattern or matrix or in one or more rows and/or columns), as shown in FIGS. 4 and 6.

In some embodiments, the consolidated view can be visually presented on a user interface (e.g., a user interface of input/output interface 113) that allowed a user to further interact with the consolidated view and/or B-scan portions corresponding to one or more patches in the consolidated view. In some implementations, the user interface can include a set of controls to view and manipulate the imaging data, analyzed data, and/or the one or more consolidated view(s) (e.g., stitched views of patches including suspicious areas detected by the data analyzer 115). The data visualizer 116, based on inputs received from the user via the set of controls, can be configured to perform any suitable image manipulation, image segmentation, and or image processing function, to aid in visual review of the imaging data. For example, a user can provide inputs via the set of controls to perform manipulations such as zooming, panning, overlaying, etc. In some embodiments, the set of control can provide options to the user to perform advanced processing functions, such as, for example, contour detection, foreground/background detection, distance or length measurements, etc. In some implementations, the data visualizer 116 can be configured to link portions of the consolidated view (e.g., stitched view) to portions of the entire imaging dataset (e.g., B-scans) to enable to user to jump between viewing the consolidated view and the B-scan including a patch that was included in the consolidated view. For example, the user interface can present a first screen with a stitched panoramic view, and upon user selection of a particular patch in the stitched panoramic view, present a second screen with the B-scan that included the selected patch. This second screen with the B-scan can enable a user to confirm a diagnosis of the tissue based on viewing the patch.

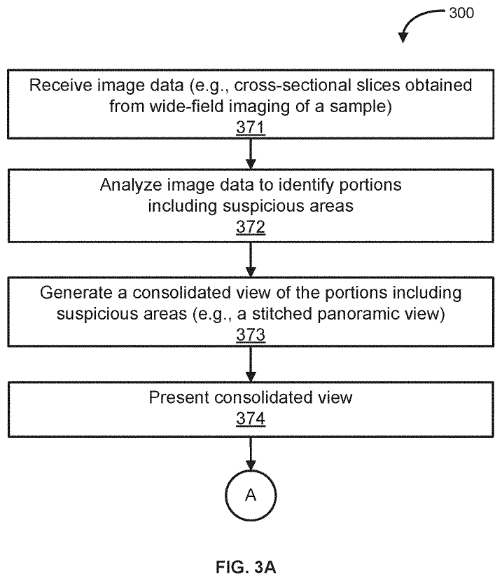

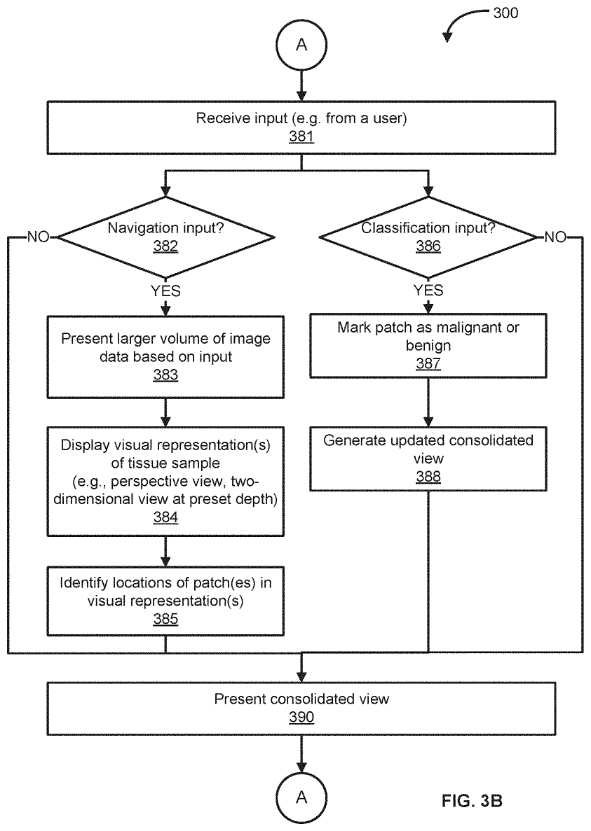

FIGS. 3A-3B illustrates an example method 300 of analyzing and/or processing image data (e.g., image dataset(s) 117) associated with a tissue portion, and presenting a visual interface of a consolidated view (e.g., stitched view(s) 119) of suspicious areas. The method 300 can be performed by a compute device, such as the compute device 101 described with reference to FIG. 2.

At 371, a compute device receives image data, e.g., including cross-sectional slices obtained from an imaging system such as a wide field OCT system. The image data can be obtained from imaging a tissue portion (e.g., biological tissue sample). In some embodiments, the image data can be obtained using an imaging system, as described in the '767 Publication. For example, such a system can include a sample container configured to support a biological tissue sample during imaging and an imaging device for generating optical images of the biological tissue sample. The sample container can be configured to interface with the imaging device such that the imaging device can image the biological tissue sample through an imaging window of the sample container, where the imaging window is partially transparent to light emitted by the imaging device. The imaging device can include a light source that emits light towards the sample and a detector configured to receive and detect light reflected from the sample. In some embodiments, the imaging device can be an OCT system, and the image data can include B-scans (i.e., cross-sectional views) of the tissue sample.

At 372, the compute device (e.g., via the processor 111 that operates as data analyzer 115) analyzes the image data to identify portions of the image data (e.g., patches of the slices) that include one or more suspicious areas. In some implementations, the compute device can implement an image processing algorithm (e.g., machine learning tool or statistical tool) to perform computer-assisted detection of the suspicious areas. For example, the compute device can be configured with a set of image features that have been validated as being indicative of the presence of tumorous tissue (e.g., though histopathological studies). The compute device can use these image features as reference to identify a set of patches that include areas suspected of being tumorous. In some implementations, the compute device can use a CAD algorithm that has been trained to classify patches of image data into different classes or categories, e.g., benign and malignant. In some implementations, the compute device can receive input from a user that further aids in its analysis and/or classification (e.g., including identification of classes, identification of areas of interest, etc.).

In some embodiments, the compute device can be configured to employ a convolutional neural network (CNN) to identify abnormal tissue in the image data, such as that described in International Patent Application No. PCT/CA2019/051532, published as International Patent Application Publication No. WO 2020/087164 (“the '164 Publication”), filed Oct. 29, 2019, and titled “Methods and Systems for Medical Image Processing Using a Convolutional Neural Network (CNN),” incorporated herein by reference. For example, the compute device can use a CNN that has a symmetric neural network architecture including (1) a first half of layers for extracting image features, reducing the feature map size, and retrieving the original image resolution, and (2) a second half of layers for identifying the likely regions of interest (ROIs) in the image data that are associated potential anomalies.

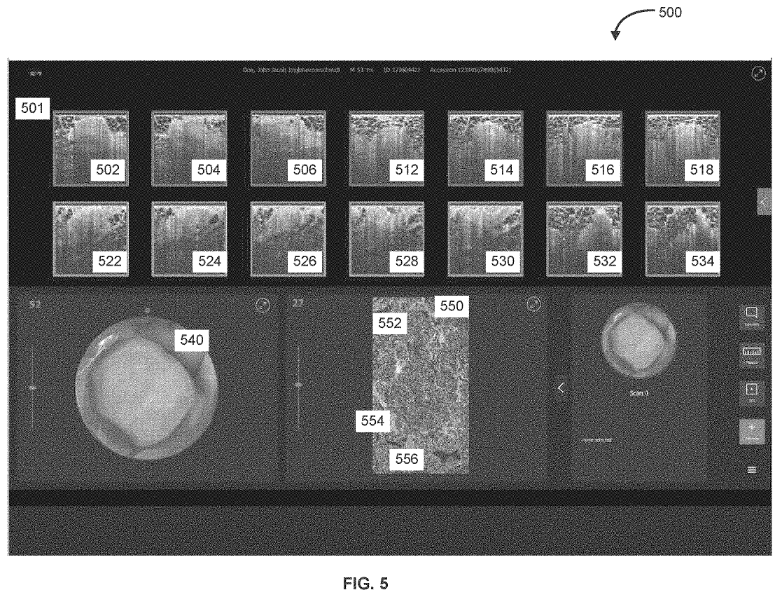

At 373, the compute device (e.g., via the processor 111 that operates as data visualizer 116) generates a consolidated view (e.g., a stitched view of 2D patches of images or stitched panoramic view, such as stitched panoramic view 119) of the portions of the image data (e.g., patches of slices) detected to include suspicious area(s) or feature(s). In some implementations, the compute device can generate a user interface that facilitates review of the suspicious patches and scans. At 374, the compute device presents, e.g., via a visual display (e.g. a display of an external device operatively coupled to the compute device 101, or a display forming part of the input/output interface 113), the consolidated view (e.g., stitched panoramic view). In some embodiments, as further described below with reference to FIGS. 5-7, the consolidated view can use various markers to group different patches and/or present detailed information associated with each patch or 2D image. In some implementations, the user interface can include a set of controls that can be used to manipulate the image data and/or the consolidated view to perform any suitable image manipulation and/or image processing function, as described above with reference to the data analyzer 116.

FIG. 3B depicts a flowchart of visualizing the image data, e.g., on a visual interface associated with compute device 101 or a compute device operatively coupled to compute device 101. At 381, the compute device can optionally receive an input, e.g., from a user providing an input into the user interface or from a separate process (e.g., implemented on the compute device 101 or an external compute device). The input can include, for example, a keyboard input, a touchscreen input, a mouse input, an alpha-numeric input, a signal, etc. In some embodiments, the input can be associated with navigating between different views of patches (e.g., 2D image data of a tissue sample) or the tissue sample. In some embodiments, the input can be associated with classifying and/or diagnosing tissue associated with one or more patches.

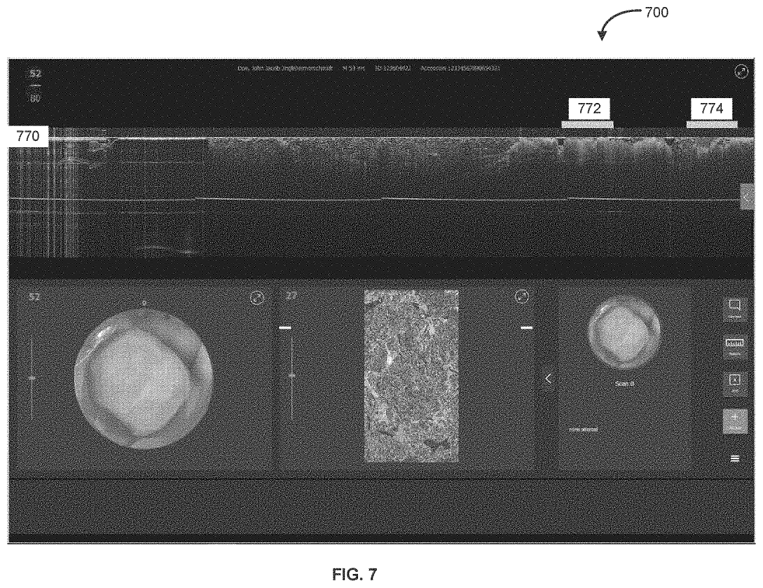

At 382, the compute device can determine whether the input is a navigation input. For example, the input can request that certain views of the tissue sample be displayed and/or a larger volume of patches from the image data be displayed. In some embodiments, the input can indicate a selection of a set of one or more patches presented in the consolidated view of diagnostically relevant portions of image data (e.g., patches having features associated with abnormal or suspicious tissue, or patches identified as having a ROI). At 383, in response to receiving a selection of a set of one or more patches from the consolidated view, the compute device can display a larger volume of the image data, e.g., a larger volume of patches from the original image data that includes patches surrounding the selected patches (i.e., patches showing portions of tissue that are spatially close to the selected patches) or a view of a larger portion of the tissue. A user, for example, can navigate between the consolidated view presented at 374 or 390 and the larger volume of image data presented at 383, e.g. by selecting various patches and/or providing inputs into the user interface (e.g., selecting or clicking on an icon or patch, selecting or clicking a region of a screen, swiping, etc.). Further details of such navigation are described with reference to FIGS. 6 and 7.

The compute device can optionally display other visual representations of the tissue sample, including different 2D and/or 3D views of the tissue sample, at 384. For example, as further described with reference to FIGS. 5 and 7, the compute device can display one or more of a perspective view of the tissue sample and/or a view of the tissue sample at a preset depth. In some embodiments, the compute device display these views based on one or more inputs, e.g., from a user. For example, a user can input a selected depth, and the compute device can display a view of the tissue sample at that selected depth. In some embodiments, as depicted in FIGS. 5 and 7, the user interface can include a plurality of portions that each display a different view of the tissue sample, including portions that display the consolidated view of diagnostically relevant image patches, perspective views of the tissue, or larger 2D scans of the tissue at different depths and/or along different directions. At 385, the compute device optionally can display one or more visual markings (e.g., different colors, symbols, text, line patterns, etc.) to identify patches that are spatially close to one another, as further described below with respect to FIGS. 5 and 6. Similarly, the compute device optionally can use visual markings to link locations in different views of the tissue sample to one another and to the image patches. For example, image patches marked with a first color, symbol, or text can be associated with a particular location in a perspective view of a tissue sample or a larger 2D scan of a tissue sample using the same color, symbol, or text. Further details of such implementations are described with reference to FIGS. 5-7.

While FIGS. 3A and 3B depict an example method 300 as including one or more events and/or steps, any one of the events and/or steps can be optionally performed or omitted. For example, events and/or steps associated with displaying different views of a tissue sample (e.g., 384) and/or identifying locations of patches (e.g., 385) can be optionally performed. Additionally or alternatively, various events and/or steps can be performed in the absence of other events and/or steps. For example, events and/or steps associated with displaying different views of a tissue sample (e.g., 384) and/or identifying locations of patches (e.g., 385) can be performed in the absence of receiving an input (e.g., 381).

FIG. 4 illustrates an example consolidated view of image data, implemented as a stitched view 419, e.g., generated using compute device(s) as described herein (e.g., compute device 101). The stitched view 419 includes portions of the image data (e.g., patches of B-scans) that have been identified as suspicious. The stitched view 419 can be of a case with a positive margin for cancer.

As depicted, the stitched view 419 includes 37 patches of tissue that were stitched (e.g., combined) together. While 37 patches with a specific fixed size are depicted, it can be appreciated that any number of patches and/or sizes of patches can be used to generate a stitched view. Below each patch of image data, the spatial location of that path can be provided. Alternatively, this information can be displayed at other locations relative to each patch (e.g., above, adjacent to, etc.). In some embodiments, other information, such as malignancy scores (e.g., DCIS score), confidence values, spatial location (e.g., location in B-scan), etc., can also be displayed near each patch to further assist a user in reviewing the image data for potential malignancy.

With the spatial location of any particular patch in the image data being known (e.g., which B-scan or portion of a B-scan), a user can easily navigate to these locations in the image data (e.g., to a complete B-scan), e.g., using the user interface and/or manually, to confirm the diagnosis. Since a diagnostic decision on the margin can be taken based on a single detection that is deemed positive, the user interface can enable faster identification of a positive or negative tissue portion and substantially decrease the time required for image review. For example, when presenting the image data, a window in the display can be populated by patches identified as suspicious (e.g., by a CAD algorithm) after acquiring each B-scan, and a user reviewing the image data can stop the scanning and analysis process once a definitive diagnostic decision has been made.

In some embodiments, systems, devices, and methods disclosed herein can include provisions for navigating between a stitched view of patches of diagnostically relevant portions of image data (e.g., patches including pathological tissue markers and/or flagged as being suspicious) and a larger volume of the image data (e.g., image dataset 117 including the original scanned volume of image data). For example, such systems, devices, and methods can provide a user interface that displays patches flagged as being diagnostically relevant with features for navigating between those patches and larger volumes of image data, e.g., to facilitate further visual review.

FIG. 5 depicts an example view of a user interface 500, including a first portion including a stitched view 501 of patches of diagnostically relevant portions of image data (e.g., patches flagged as suspicious for DCIS), a second portion including a top perspective view of a portion of tissue 540, and a third portion including a view of an entire volume 550 of image data shown at a preset depth. As depicted, sets of patches of the stitched view 501 can be taken from different locations in the entire volume 550, and can be color coded different colors. For example, a first set of patches 502, 504, 506 can be color coded red and be taken from a first location in the volume 550; a second set of patches 512, 514, 516, 518 can be color coded blue and be taken from a second location in the volume 550; a third set of patches 522, 524, 526, 528, 530 can be color coded green and be taken from a third location in the volume 550, and a fourth set of patches 532, 534 can be color coded purple and be taken from a fourth location in the volume 550. In the view of the volume 550, similarly colored markings 552, 554, 556 can be used to indicate a general location of the sets of patches. For example, the location of the first set of patches 502, 504, 506 in the volume 550 can be indicated using a red marking 552; the location of the second set of patches 512, 514, 516, 518 in the volume 550 can be indicated using a blue marking 554; and so on and so forth. By color coding the sets of patches, as well as their locations in the entire volume, the user interface 500 facilitates with mapping the patches flagged as being diagnostically relevant in the stitched view 501 to their location in the originally scanned volume 550.

While different colors are used to associate the various sets of patches with one another (e.g., first set of patches 502, 504, 506, and second set of patches 512, 514, 516, 518), it can be appreciated that other characteristics and/or types of markings can be used to associated sets of patches with one another, and with their spatial location(s) in a tissue sample. For example, a letter (e.g., “A” or “B”) displayed proximate to one or more patches can be used to associate those patches with one another, while that same letter can be displayed in a perspective view of the tissue sample at the general spatial location corresponding to those patches. Other examples of suitable markings include symbols, characters, line patterns, highlighting, etc.

FIG. 6 is an example view of a user interface 600, showing a stitched view 601 of patches of imaged areas flagged as diagnostically relevant, e.g., suspicious for DCIS. FIG. 6 can provide a stitched view 601 that includes a larger number of patches than those depicted in FIG. 5. In an embodiment, FIG. 6 can include the patches depicted in FIG. 5 as a first group of patches 602 and can include additional patches included in a second group of patches 610. As described with reference to FIG. 5, patches from groups 602 and/or 610 can belong to different sets that are each taken from different locations in a larger volume of image data. In an embodiment, different sets from different locations can be color coded different colors (e.g., red, blue, green, purple, etc.).

In some embodiments, the patches in the stitched view 601 can be interactive. For example, a user can select a particular patch 612 (or any other patch) and, in response to receiving such selection, the user interface 600 can change to show a view of the two-dimensional scan that includes the selected patch 612. More specifically, a processor (e.g., processor 111) controlling user interface 600 can be configured to, in response to receiving the selection by a user of a patch 612, determine the two-dimensional scan (e.g., B-scan) from the larger three-dimensional stack or volume of imaging data that includes the patch 612 and cause the user interface 600 to display at least a portion of that two-dimensional scan.

For example, by selecting one patch 612, e.g., indicated by a cursor 660, a user can navigate to two-dimensional scan including that patch 612 for further visual inspection (e.g., inspection of adjacent areas, etc.) to, for example, further assess if the flagged patch depicts an area that is potentially a true positive, e.g., for one or more suspicious markers, or is a negative and can be omitted from further analysis. FIG. 7 depicts an example view of a user interface 700 showing a two-dimensional scan 770 from the larger volume of imaging data, such as, for example, one that a user can be navigated to by selecting the patch 612 in FIG. 6. The two-dimensional scan 770 shown in FIG. 7 can include portions marked with tags 772, 774 that are colored, e.g., to correspond to the color of the patch (e.g., patch 612) that the user had selected. In this example, the color of the patch 612 and the color of the tags 772, 774 are yellow.

While various embodiments have been described above, it should be understood that they have been presented by way of example only, and not limitation. Where methods and/or schematics described above indicate certain events and/or flow patterns occurring in certain order, the ordering of certain events and/or flow patterns may be modified. While the embodiments have been particularly shown and described, it will be understood that various changes in form and details may be made.

Although various embodiments have been described as having particular features and/or combinations of components, other embodiments are possible having a combination of any features and/or components from any of embodiments as discussed above.

Some embodiments described herein relate to a computer storage product with a non-transitory computer-readable medium (also can be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also can be referred to as code) may be those designed and constructed for the specific purpose or purposes. Examples of non-transitory computer-readable media include, but are not limited to, magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical disks; carrier wave signal processing modules; and hardware devices that are specially configured to store and execute program code, such as Application-Specific Integrated Circuits (ASICs), Programmable Logic Devices (PLDs), Read-Only Memory (ROM) and Random-Access Memory (RAM) devices. Other embodiments described herein relate to a computer program product, which can include, for example, the instructions and/or computer code discussed herein.

In this disclosure, references to items in the singular should be understood to include items in the plural, and vice versa, unless explicitly stated otherwise or clear from the context. Grammatical conjunctions are intended to express any and all disjunctive and conjunctive combinations of conjoined clauses, sentences, words, and the like, unless otherwise stated or clear from the context. Thus, the term “or” should generally be understood to mean “and/or” and so forth. The use of any and all examples, or exemplary language (“e.g.,” “such as,” “including,” or the like) provided herein, is intended merely to better illuminate the embodiments and does not pose a limitation on the scope of the embodiments or the claims.

Some embodiments and/or methods described herein can be performed by software (executed on hardware), hardware, or a combination thereof. Hardware modules may include, for example, a general-purpose processor, a field programmable gate array (FPGA), and/or an application specific integrated circuit (ASIC). Software modules (executed on hardware) can be expressed in a variety of software languages (e.g., computer code), including C, C++, Java™, Ruby, Visual Basic™, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. For example, embodiments may be implemented using imperative programming languages (e.g., C, Fortran, etc.), functional programming languages (Haskell, Erlang, etc.), logical programming languages (e.g., Prolog), object-oriented programming languages (e.g., Java, C++, etc.) or other suitable programming languages and/or development tools. Additional examples of computer code include, but are not limited to, control signals, encrypted code, and compressed code.

1. An apparatus, comprising:
a display configured to display a user interface;
a memory; and
a processor operatively coupled to the display and the memory, the processor configured to:
receive image data of a tissue sample;
identify, using a machine learning algorithm trained using a set of images to identify regions of abnormality in the image data, a set of patches of the image data, each patch from the of patches including at least one feature associated with an abnormality;
generate a consolidated view of the set of patches in which the set of patches are arranged according to a predefined layout; and
display the consolidated view of the set of patches on the user interface; and
wherein each patch from the set of patches is associated with a spatial location in the tissue sample and the processor is configured to display markings in the consolidated view to associate patches from the set of patches that have proximate spatial locations with one another.

2. The apparatus of claim 1, wherein the image data includes a set of two-dimensional scans of the tissue sample,
the processor further configured to process the set of two-dimensional scans of the tissue sample into a plurality of patches each having the same predefined dimensions, each patch from the plurality of patches being a different portion of a two-dimensional scan from the set of two-dimensional scans,
the set of patches being patches from the plurality of patches that include at least one feature associated with an abnormality.

3. (canceled)

4. The apparatus of claim 1, wherein the processor is configured to display (1) a first marking having a first color proximate to each patch from a first subset of the set of patches and (2) a second marking having a second color different from the first color proximate to each patch from a second subset of the set of patches,
each patch from the first subset of patches having a spatial location that is adjacent to at least one other patch from the first subset of patches, and each patch from the second subset of patches having a spatial location that is adjacent to at least one other patch from the second subset of patches.

5. The apparatus of claim 1, wherein the processor is configured to display the consolidated view of the set of patches in a first area of the user interface,
the processor further configured to display a perspective view of the tissue sample in a second area of the user interface such that the perspective view of the tissue sample is displayed together with at least a portion of the consolidated view of the set of patches.

6. The apparatus of claim 5, wherein the processor is further configured to display a two-dimensional view of the tissue sample at a predefined depth in a third area of the user interface such that the two-dimensional view of the tissue sample is displayed together with the perspective view and at least a portion of the consolidated view of the set of patches.

7. The apparatus of claim 6, wherein each patch from the set of patches is associated with a spatial location in the tissue sample,
the processor further configured to display a first marking proximate to a patch from the set of patches in the consolidated view and a second marking in at least one of the perspective view or the two-dimensional view that is indicative of the spatial location of the patch, the first and second markings sharing a common characteristic.

8. The apparatus of claim 7, wherein the first and second markings have the same color.

9. The apparatus of claim 6, wherein the processor is configured to, in response to receiving an input indicating a different predefined depth, display a two-dimensional view of the tissue sample at the different predefined depth.

10. The apparatus of claim 1, wherein the processor is configured to, in response to detecting a selection of a patch from the set of patches, display a two-dimensional scan of the tissue sample that includes the patch selected from the set of patches.

11. The apparatus of claim 10, wherein the processor is further configured to display a marking proximate to a portion of the two-dimensional scan that corresponds to a location of the patch selected from the set of patches.

12. The apparatus of claim 1, wherein the processor is further configured to, in response to receiving an input indicating that a patch from the set of patches is not associated with an abnormality:
remove the patch from the set of patches; and
after removing the patch from the set of patches, generate an updated consolidated view of the set of patches.

13. The apparatus of claim 1, further comprising an imaging device configured to obtain the image data,
the processor operatively coupled to the imaging device.

14. An apparatus, comprising:
a memory; and
a processor operatively coupled to the memory, the processor configured to:
receive a set of two-dimensional image scans of a three-dimensional tissue sample;
process the set of two-dimensional image scans to produce a set of patches each having the same dimensions, each patch from the set of patches including image data from a different portion of the set of two-dimensional image scans;
identify, using a machine learning algorithm trained using a set of images to identify regions of abnormality in the image data, a subset of patches (1) from the set of patches and (2) including at least one feature associated with an abnormality; and
generate a consolidated view of the subset of patches in which the subset of patches are arranged according to a predefined layout, and wherein each patch from the set of patches is associated with a spatial location in the tissue sample, the subset of patches arranged in the consolidated view such that patches from the subset of patches having spatial locations proximate to one another are arranged adjacent to one another.

15. (canceled)

16. The apparatus of claim 14, wherein the consolidated view of the subset of patches includes information associated with each patch from the subject of patches, the information including at least one of: a ductal carcinoma in situ (DCIS) score, a confidence value associated with the DCIS score, spatial location information.

17. The apparatus of claim 14, wherein the processor is further configured to, in response to receiving an input indicating that a patch from the subset of patches is not associated with an abnormality:
remove the patch from the subset of patches; and
after removing the patch from the subset of patches, generate an updated consolidated view of the subset of patches.

18. A method, comprising:
receiving image data of a tissue sample;
identifying, using a machine learning algorithm trained using a set of images to identify regions of abnormality in the image data, a set of patches of the image data, each patch from the of patches including at least one feature associated with an abnormality;
generating a consolidated view of the set of patches in which the set of patches are arranged according to a predefined layout; and
displaying the consolidated view of the set of patches on a user interface and wherein each patch from the set of patches is associated with a spatial location in the tissue sample, the method further comprising: displaying markings in the consolidated view to associate patches from the set of patches that have proximate spatial locations with one another.

19. (canceled)

20. The method of claim 18, wherein the consolidated view of the set of patches is displayed in a first area of the user interface, the method further comprising:
displaying in one or more second areas of the user interface at least one of: a perspective view of the tissue sample, or a two-dimensional view of the tissue sample at a predefined depth.

21. The apparatus of claim 14, wherein the processor is configured to display the consolidated view of the set of patches in a first area of the user interface, the processor further configured to display a perspective view of the tissue sample in a second area of the user interface such that the perspective view of the tissue sample is displayed together with at least a portion of the consolidated view of the set of patches.

22. The apparatus of claim 14, wherein the processor is further configured to display a two-dimensional view of the tissue sample at a predefined depth in a third area of the user interface such that the two-dimensional view of the tissue sample is displayed together with the perspective view and at least a portion of the consolidated view of the set of patches.

23. The apparatus of claim 14, wherein each patch from the set of patches is associated with a spatial location in the tissue sample, the processor further configured to display a first marking proximate to a patch from the set of patches in the consolidated view and a second marking in at least one of the perspective view or the two-dimensional view that is indicative of the spatial location of the patch, the first and second markings sharing a common characteristic.
